(19) United States
(12) Patent Application Publication
(10) Pub. No.: US 2026/0060770 A1
(43) Pub. Date: Mar. 5, 2026

(54) BUTTERFLY BASE WORKSTATION ON WHEELS

(71) Applicant: Arizona Board of Regents on Behalf of Arizona State University, Scottsdale, AZ (US)

(72) Inventors: Erika GRIFFIN, Phoenix, AZ (US); Michael COLLINS, Glendale, AZ (US); Darren GROZIER, Phoenix, AZ (US); David GROZIER, Peoria, AZ (US); Karanraj GOVINDRAJ, Tempe, AZ (US); Tanvi WAKNIS, Phoenix, AZ (US); Colin STAFFORD, Spring, TX (US); Anushka MITBANDER, Chandler, AZ (US); Wissem Fadia BABAGHAYOU, Tempe, AZ (US); Sheetal JHA, Peoria, AZ (US); Savannah REDMOND, Phoenix, AZ (US); Sasha SANTAMARIA, Phoenix, AZ (US); Abigail BAKER, Phoenix, AZ (US); Tanvi SONAWALE, Tempe, AZ (US); Angelica CASAS, Phoenix, AZ (US); Jarizbeth GUERRERO MENDIVIL, Mesa, AZ (US)

(21) Appl. No.: 19/318,267

(22) Filed: Sep. 3, 2025

Related U.S. Application Data
(60) Provisional application No. 63/690,272, filed on Sep. 3, 2024.

(51) Int. Cl. A61B 50/13; A47B 21/02; A47B 21/06; A61B 50/18

(52) U.S. Cl. CPC A61B 50/13; A47B 21/02; A61B 50/18; A47B 2021/066; A61B 2050/185

(57) ABSTRACT

A mobile workstation configured to enhance workflow and accessibility is disclosed. The workstation includes a base that supports and stabilizes the unit in an upright position and may have a butterfly-style shape. A rechargeable battery is positioned in the base and powers the workstation, which is configured for battery replacement without turning off the workstation. The base may comprise a base light to illuminate surrounding areas and may be configured to slide partially beneath objects such as a hospital beds. A stem extends from the base, comprising a lower portion, a storage attachment that may include compartments for medical supplies and a storage light, and an upper portion. A tabletop is positioned on the upper portion and may support a monitor with a table light. A keyboard tray may be suspended below the tabletop and may include a keyboard light. The monitor's position may be adjustable.

### RELATED APPLICATIONS

This application claims the benefit of U.S. provisional patent application 63/690,272, filed Sep. 3, 2024, to Griffin, et al., titled “U-Shaped, Butterfly Base Workstation on Wheels,” the entirety of the disclosure of which is hereby incorporated by this reference.

### TECHNICAL FIELD

This document relates to a mobile workstation for healthcare professionals.

### BACKGROUND

A mobile workstation, or workstation on wheels (WoW), is a crucial device for nurses to have in a healthcare setting. Nurses often have hectic schedules, long shifts, and physically demanding work which requires them to handle patient records, medications, and medical supplies in an efficient manner. WoWs enable nurses to have all necessary tools and documentation at their fingertips, which facilitates better patient care and improves workflow. In an environment where every second counts, the mobility and accessibility of a WoW is essential for reducing time spent retrieving supplies, which increases the time available for direct patient care.

However, traditional WoWs present several challenges that make them hard to work with and exacerbate the already demanding nature of nursing. These challenges include limited mobility due to cumbersome designs, insufficient storage options that do not securely hold essential supplies, and the frequent need to recharge electronic components. The design of most contemporary WoWs does not account for the increasingly compacted and crowded spaces of modern healthcare facilities, making them difficult to maneuver and use effectively. These issues contribute to inefficiencies and can increase the physical strain on nurses, leading to fatigue, discomfort, and burnout.

As mentioned above, the inefficient design and limited maneuverability oftentimes contribute to nurse burnouts which are alarmingly high, with approximately 40% of nurses reporting severe burnout. Inefficient workstations are a major contributing factor, leading to about 30% of nurses experiencing musculoskeletal injuries related to their work environment. Additionally, inadequate WoW designs lead to the improper handling and storage of medical supplies which contributes to higher infection rates in healthcare settings. Patient feedback also frequently indicates that approximately 50-60% of patient-nurse interactions are perceived as hindered because nurses are often behind screens or busy with computers. These statistics point toward the urgent need for an improved mobile workstation that can alleviate these issues.

### SUMMARY

The present disclosure is related to a mobile workstation, or workstation on wheels (WoW), that is designed to improve workflow, ergonomics, and accessibility for healthcare professionals. Embodiments of the present disclosure feature novel bed-hugging technology in the form of a U-shaped, butterfly-style base that is connected to an extendable stem with a flowing tabletop, which enables the device to seamlessly slide beneath objects such as hospital beds. The workstation may also include versatile storage options and cordless electronic attachments supported by a long-lasting battery life, achieved by integrating rechargeable, hot-swappable batteries. Traditional WoWs often pose challenges such as limited mobility, hefty structure, and frequent recharging needs, leading to inefficiencies and physical strain for nurses. The primary objective of the present disclosure is to address these challenges by providing a compact, ergonomic, and efficient mobile workstation that minimizes physical strain, optimizes space in compact medical care facilities, and enhances overall efficiency in patient care tasks. This streamlined, provider-friendly solution aims to support healthcare professionals in the delivery of optimal patient care, improve daily operations within healthcare environments, contribute to better patient outcomes, and increase productivity while minimizing burnout, injuries, and fatigue due to mobile workstation use. The scope of the present disclosure, even though specific embodiments are designed to be used by healthcare workers, is not limited to healthcare-specific settings.

According to some embodiments, a mobile workstation comprises a base configured to support and stabilize the mobile workstation in an upright position, wherein the base has a butterfly-style shape and comprises a base light configured to illuminate the surroundings of the base, an extendable stem comprising a lower portion extending up from the base offset towards a front edge of the base, a storage attachment mounted on the lower portion, the storage attachment having a plurality of compartments configured to store medical supplies and at least one storage light configured to illuminate an interior of the plurality of compartments, and an upper portion extending up from the storage attachment angled away from the front edge of the base, a tabletop positioned on the upper portion of the extendable stem, a monitor extending up from the tabletop and having a table light configured to illuminate the tabletop, a keyboard tray suspended below the tabletop configured to support a keyboard operatively coupled to the monitor, and a rechargeable battery positioned in the base and configured to power the workstation, wherein the workstation is configured for battery replacement without turning off the workstation.

Particular embodiments may comprise one or more of the following features. The workstation may be designed with minimal crevices. The workstation may comprise a keyboard light configured to illuminate the keyboard. The base may be configured to slide at least partially beneath a hospital bed. The upper portion of the extendable stem may be angled such that when the base slides beneath the hospital bed, the tabletop is positioned above the hospital bed. A position of the monitor with respect to the tabletop may be adjustable.

According to some embodiments, a mobile workstation comprises a base configured to support and stabilize the mobile workstation in an upright position, wherein the base has a butterfly-style shape, an extendable stem comprising a lower portion extending up from the base offset towards a front edge of the base, a storage attachment mounted on the lower portion, the storage attachment having a plurality of compartments configured to store medical supplies, and an upper portion extending up from the storage attachment angled away from the front edge of the base, a tabletop positioned on the upper portion of the extendable stem, a monitor extending up from the tabletop, and a rechargeable battery positioned in the base and configured to power the workstation, wherein the workstation is configured for battery replacement without turning off the workstation.

Particular embodiments may comprise one or more of the following features. The base may comprise a base light configured to illuminate the surroundings of the base. The base light may be configured to turn on when an ambient light level lowers past a predetermined threshold. The mobile workstation may further comprise a keyboard tray suspended below the tabletop configured to support a keyboard operatively coupled to the monitor. The storage attachment may comprise a glove box holder mounted to an outer surface of the storage attachment. The base may be configured to slide at least partially beneath a hospital bed. The upper portion of the extendable stem may be angled such that when the base slides beneath the hospital bed, the tabletop is positioned above the hospital bed.

According to some embodiments, a mobile workstation comprises a base configured to support and stabilize the mobile workstation in an upright position, a stem extending up from the base offset towards a front edge of the base, a tabletop positioned on top of the stem, a monitor extending up from the tabletop, and a rechargeable battery positioned in the base and configured to power the workstation, wherein the workstation is configured for battery replacement without turning off the workstation.

Particular embodiments may comprise one or more of the following features. The stem may comprise a lower portion extending up from the base offset towards the front edge of the base, a storage attachment mounted on the lower portion, the storage attachment having a plurality of compartments configured to store medical supplies, and an upper portion extending up from the storage attachment angled away from the front edge of the base. The base may have a butterfly-style shape. The base may comprise a base light configured to illuminate the surroundings of the base. The base light may be configured to turn on when an ambient light level lowers past a predetermined threshold. The base may be configured to slide at least partially beneath a hospital bed. The stem may comprise an upper portion angled away from the front edge of the base such that when the base slides beneath the hospital bed, the tabletop is positioned above the hospital bed.

The foregoing and other aspects, features, and advantages will be apparent from the DESCRIPTION and DRAWINGS, and from the CLAIMS.

### BRIEF DESCRIPTION OF THE DRAWINGS

Implementations will hereinafter be described in conjunction with the appended and/or included DRAWINGS, where like designations denote like elements.

FIG. 1 is a perspective view of a mobile workstation according to some embodiments.

FIG. 2 is a front view of a mobile workstation according to some embodiments.

FIG. 3 illustrates the base of a mobile workstation according to some embodiments.

FIG. 4 illustrates the storage area of a mobile workstation according to some embodiments.

FIG. 5 illustrates the tabletop of a mobile workstation according to some embodiments.

FIG. 6 illustrates a monitor of a mobile workstation according to some embodiments.

FIG. 7 is a side view of the keyboard tray of a mobile workstation according to some embodiments.

FIG. 8 is a perspective view of the keyboard tray of a mobile workstation according to some embodiments.

FIG. 9 illustrates battery replacement of a mobile workstation according to some embodiments.

FIG. 10 illustrates a battery charging station for a mobile workstation according to some embodiments.

FIG. 11 is a perspective view of a mobile workstation according to some embodiments.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

Throughout the description and claims of this specification, the words “comprise” and “contain” and variations of the words, for example “comprising” and “comprises”, mean “including but not limited to”, and are not intended to (and do not) exclude other components.

As required, detailed embodiments of the present disclosure are included herein. It is to be understood that the disclosed embodiments are merely exemplary of the present disclosure that may be embodied in various forms. Therefore, specific structural and functional details disclosed herein are not to be interpreted as limits, but merely as a basis for teaching one skilled in the art to employ the present disclosure. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed subject matter. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

More specifically, this disclosure, its aspects and embodiments, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

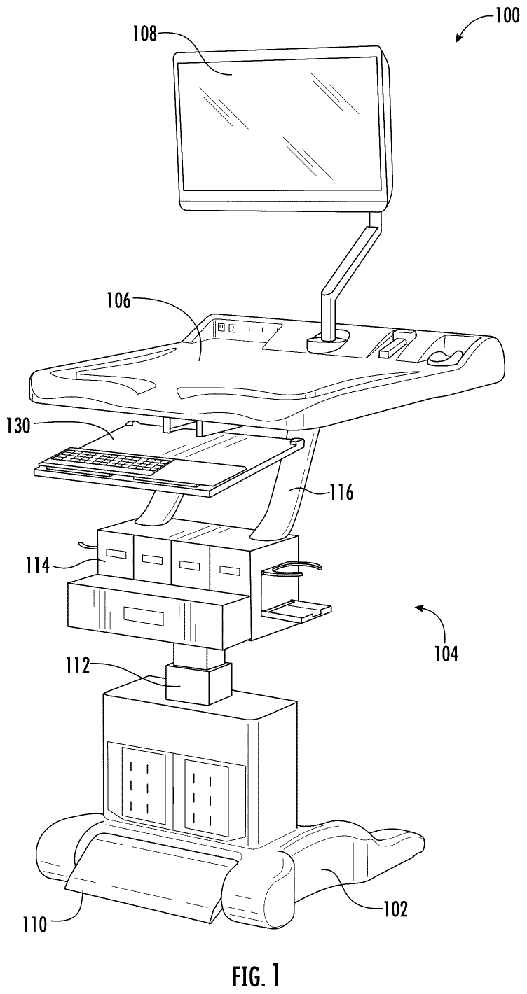

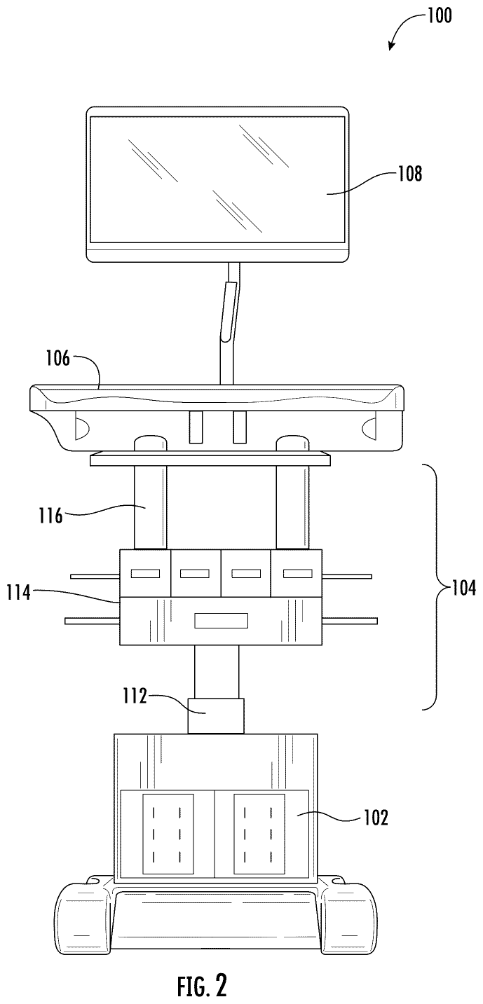

The present disclosure is related to a mobile workstation 100 developed in response to the needs described above. The mobile workstation 100 was developed to provide a more convenient and efficient option, representing a significant upgrade from traditional WoWs. In some embodiments, the mobile workstation 100 is designed for use by nurses within healthcare environments, but the mobile workstation 100 may be used by other people and/or in other environments as well. One embodiment of the mobile workstation 100 is illustrated in FIGS. 1 and 2. The mobile workstation 100 may comprise a base 102, a stem 104, a tabletop 106, and/or a monitor 108.

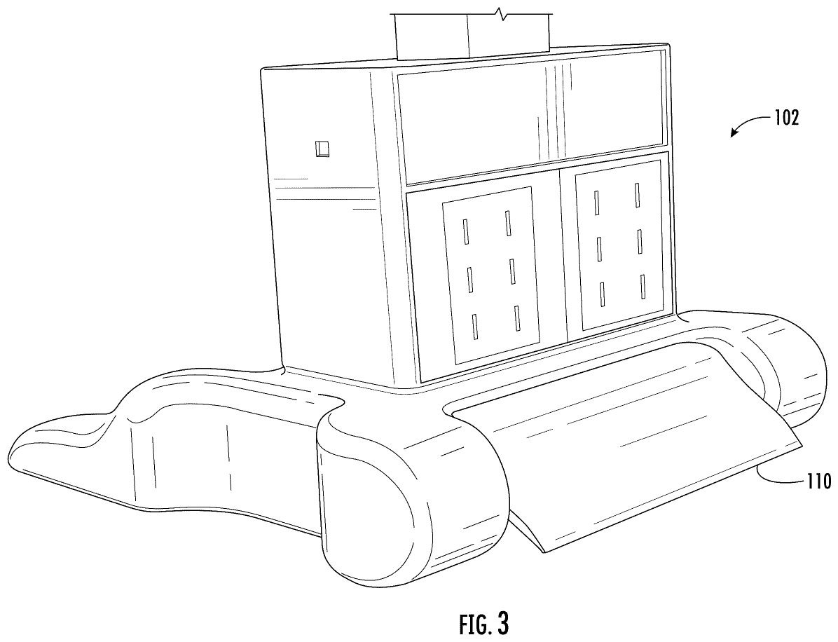

The base 102 is configured to support and stabilize the mobile workstation 100 in an upright position. As shown in FIG. 3, in some embodiments, the base 102 is U-shaped and/or has a butterfly-style shape. The base 102 has wheels that allow the base 102 to be moved easily across surfaces. In some embodiments, the base 102 is configured to slide at least partially beneath a hospital bed. For example, the U-shaped design and/or the butterfly-style design, extending away from the user or from the front edge 110 of the base 102, enable the base 102 to slide under objects like beds. This minimizes the footprint of the mobile workstation 100, making it ideal for compact settings. Existing WoWs typically have bulky designs that are difficult to maneuver in compact and crowded healthcare settings, leading to inefficiencies. In contrast, embodiments of the mobile workstation 100 that have the U-shaped, butterfly-style base 102 allow the workstation 100 to slide seamlessly beneath objects such as hospital beds. This bed-hugging design optimizes space utilization, making it ideal for crowded medical care facilities and reducing the need for frequent repositioning.

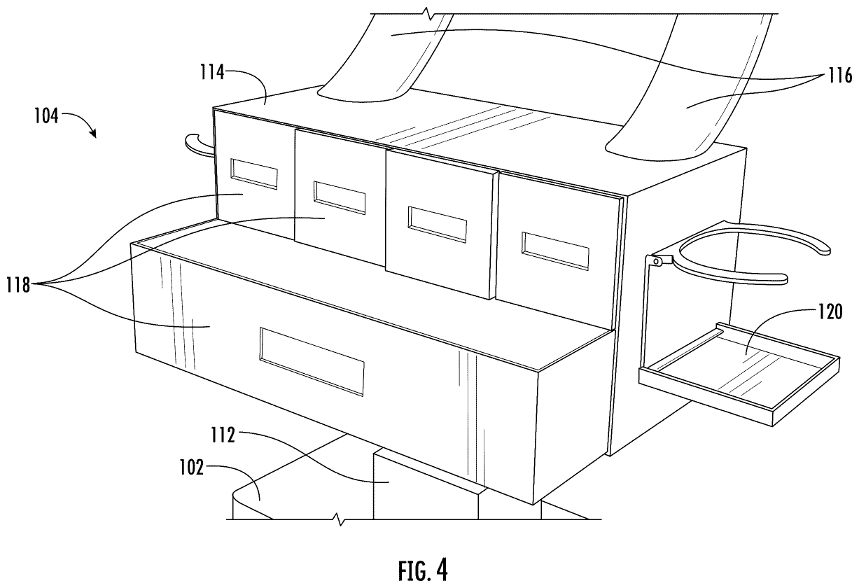

The stem 104 forms the central support structure, as shown in FIGS. 1-2. The stem 104 extends up from the base 102. In some embodiments, the stem 104 is offset towards a front edge 110 of the base 102, as shown in FIG. 1. The stem 104 may be extendable, allowing the stem 104 to be adjusted to be shorter or taller depending on the desired configuration. This allows the user to adjust the mobile workstation 100 to their height. In some embodiments, the stem 104 comprises a lower portion 112, a storage attachment 114, and/or an upper portion 116, as shown in FIG. 4. The lower portion 112 extends up from the base 102 and may be offset towards the front edge 110 of the base 102. The storage attachment 114 is mounted on the lower portion 112 between the lower portion 112 and the upper portion 116. In some embodiments, the storage attachment 114 has a plurality of compartments 118 that are configured to store medical supplies. The plurality of compartments 118 may include both lockable and open compartments, offering secure and convenient storage for medical supplies, tools, and miscellaneous items. The plurality of compartments 118 may include drawers and/or cabinets. The mobile workstation 100 may also include versatile storage options in addition to the plurality of compartments 118, such as cordless electronic attachments like a long-range scanner. Other storage options may also be included, such as a glove box holder 120 or a sanitizing wipes holder. In this way, common medical supplies such as sanitizing wipes and gloves, as well as medical tools such as stethoscopes, are readily available. In some embodiments, the glove box holder 120 is mounted to an outer surface of the storage attachment 114.

The upper portion 116 extends up from the storage attachment 114. In some embodiments, the upper portion 116 extends away from the user, or away from the front edge 110 of the base 102, in a cantilevered design. The upper portion 116 may have two support struts with a wave shape. In some embodiments, the upper portion 116 is angled away from the front edge 110 of the base 102. In some embodiments, the upper portion 116 of the stem 104 is angled such that when the base 102 slides beneath a hospital bed, the tabletop 106 is positioned above the hospital bed. Thus, the shape of the upper portion 116 of the stem 104, and in particular the cantilevered design, allow the tabletop 106 to be positioned over objects similar to beds. This enhances accessibility and usability, particularly in confined spaces typical of medical care facilities.

The tabletop 106 is positioned on the stem 104. The tabletop 106 may be positioned on the upper portion 116 of the stem 104. In some embodiments, the tabletop 106 is configured to be positioned above the hospital bed when the base 102 slides beneath the hospital bed. The mobile workstation 100 is thus designed to enhance efficiency, ergonomics, and accessibility for healthcare professionals, particularly in compact hospital settings. With the U-shaped, butterfly-style base 102 and the extendable stem 104 with a flowing tabletop 106, the design allows the mobile workstation 100 to slide seamlessly beneath objects like beds. Additionally, the cantilevered design of the upper portion 116 of the stem 104 and the tabletop 106 described above allows the tabletop 106 to be positioned over and around bed-like objects. This configuration allows the mobile workstation 100 to “hug” the bed, which saves space without requiring frequent repositioning. This design optimizes space and minimizes physical strain on the user and, most importantly, improves the quality of provider-patient interaction.

Ergonomically, many traditional WoWs have static designs that require nurses to make frequent physical adjustments, increasing strain and fatigue. The outwardly-extended tabletop 106 incorporated in the mobile workstation 100 disclosed herein allows the device's upper half to slide over and around bed-like objects as described above. This minimizes the workstation's physical footprint and enhances accessibility, which reduces the need for nurses to reach or strain, promoting better posture and reducing fatigue.

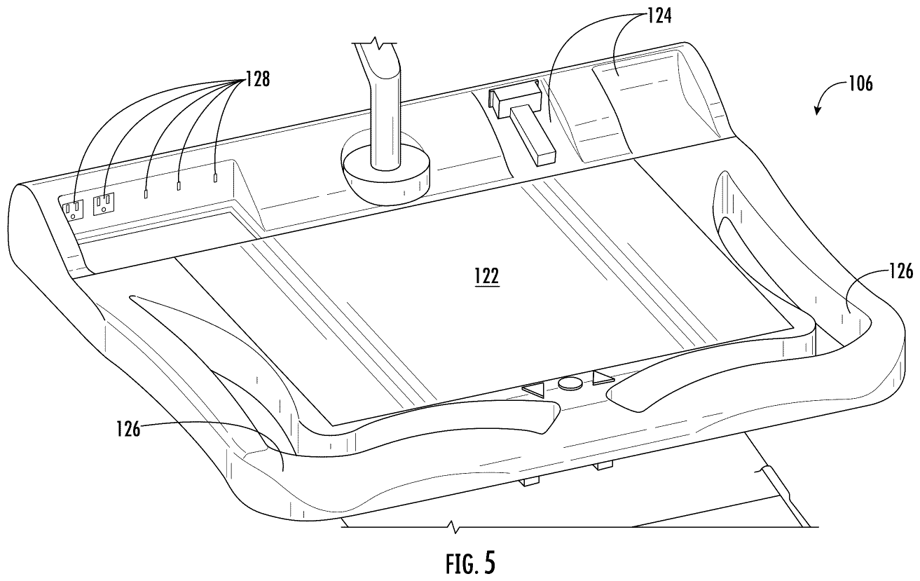

As shown in FIG. 5, the tabletop 106 may have a workspace 122 with a flat surface to allow the user to perform tasks such as writing notes and preparing medications. In addition, the tabletop 106 may have tool spaces 124 set aside to allow tools to be stored on the tabletop 106. For example, a computer mouse may be stored on the tabletop 106 in one of the tool spaces 124. The tabletop 106 may also have handles 126 on the tabletop 106 to facilitate positioning the mobile workstation 100 where desired. The handles 126 may extend around multiple sides of the tabletop 106, such as along the front and along both the right side and the left side of the tabletop 106.

The mobile workstation 100 may be configured to electrically couple with and power multiple electronic attachments. For example, as shown in FIG. 5, the tabletop 106 may have a plurality of electrical receptacles 128 that allow electrical devices or other electronic attachments to plug into the mobile workstation 100 and receive power from the mobile workstation 100. These attachments are designed to facilitate various tasks, including patient monitoring, data entry, and communication, but not limited to these functions.

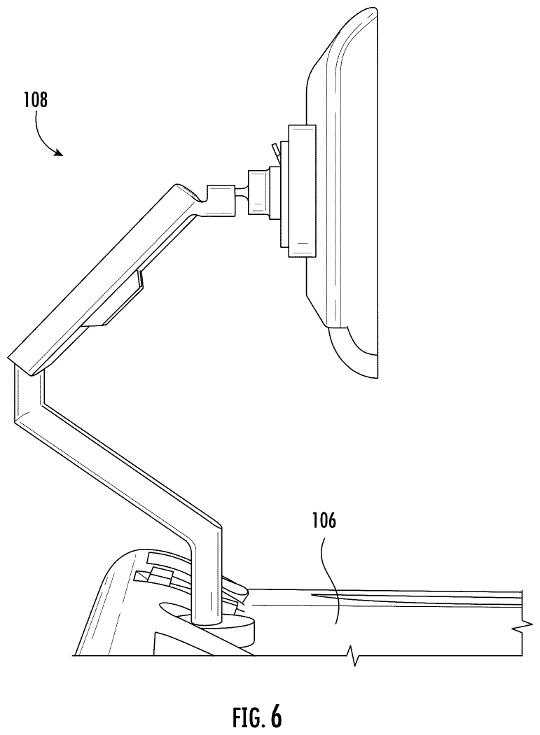

As shown in FIG. 6, the monitor 108 extends up from the tabletop 106. In some embodiments, the position of the monitor 108 with respect to the tabletop 106 is adjustable. The monitor 108 may be offset and highly-maneuverable such that it is simple for the monitor 108 to be moved to not obstruct the patient's view of the care provider. Traditional WoWs often obstruct a patient's view of healthcare workers due to centrally placed monitors, which are placed closer to the user, creating a barrier in effective communication. The offset, highly-maneuverable monitor design of some embodiments of the mobile workstation 100 ensures unobstructed nurse-patient interaction, allowing for direct eye contact and better connection. This monitor design improves communication and patient satisfaction whilst seamlessly assisting healthcare workers in their balancing act of performing digital tasks while connecting with patients.

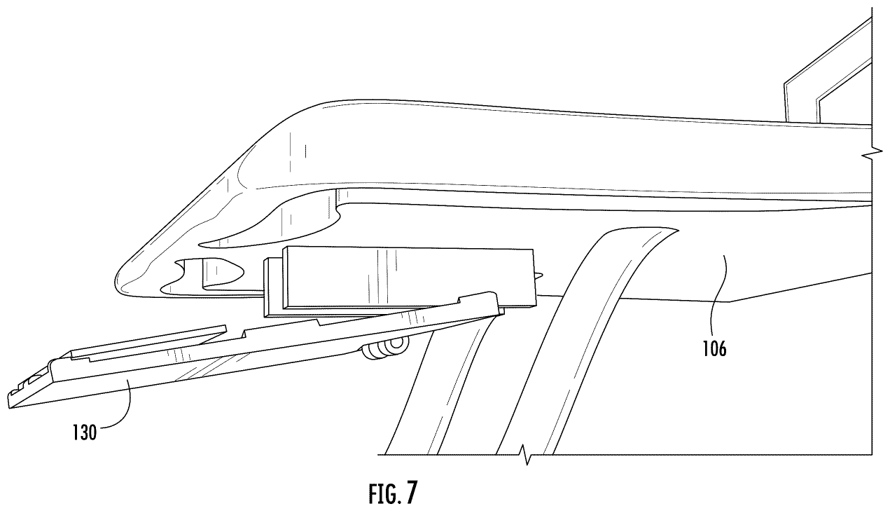

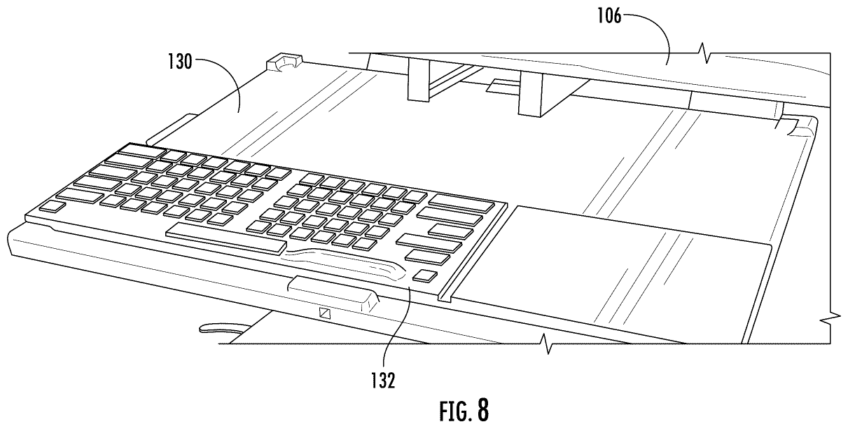

The mobile workstation 100 may also comprise a keyboard tray 130, as shown in FIGS. 7-8. The keyboard tray 130 may be suspended below the tabletop 106 and is configured to support a keyboard 132 that is operatively coupled to the monitor 108. The keyboard tray 130 may be suspended from the tabletop 106 or may be suspended from the upper portion 116 of the stem 104 to provide a comfortable location for the keyboard 132. In some embodiments, the keyboard tray 130 is attached to the tabletop 106.

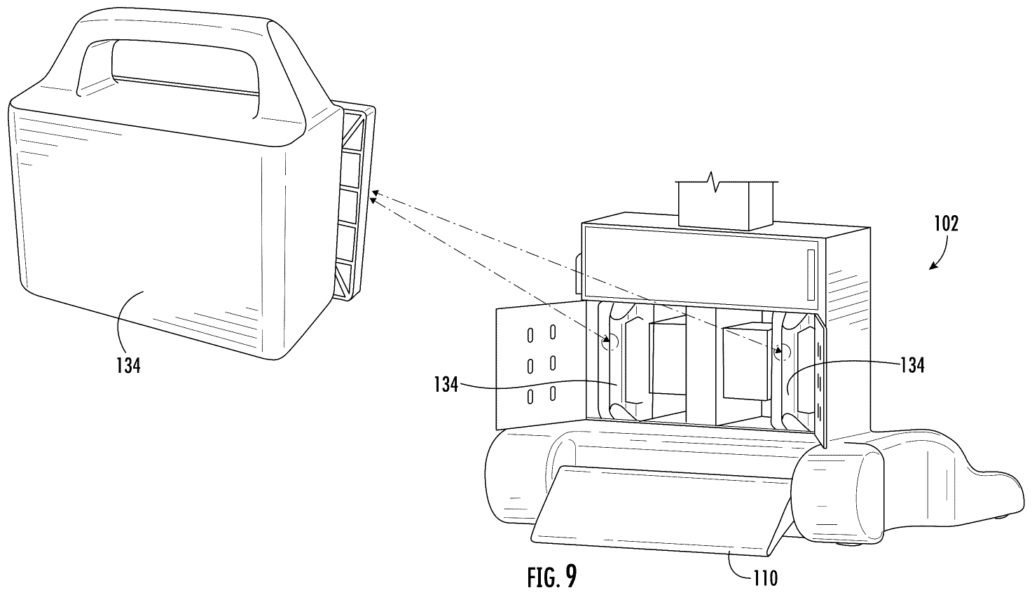

The mobile workstation 100 may be designed for cordless operation and may be powered by a long-life battery 134 that eliminates the need for frequent recharging, ensuring uninterrupted use of the electronic attachments. Thus, in some embodiments, the mobile workstation 100 comprises a battery 134 configured to power the mobile workstation 100. The battery 134 may be a rechargeable battery. As shown in FIG. 9, in some embodiments, the battery 134 is positioned and/or housed in the base 102. The long-lasting battery 134 helps to ensure uninterrupted use. In this way, the battery 134 provides an advantage over traditional WoWs. Frequent recharging of electronic components in traditional WoWs can disrupt workflow and reduce efficiency. The incorporated cordless electronic attachments are supported by a long-lasting, interchangeable battery 134, which eliminates the need for frequent recharging and helps to ensure uninterrupted usage.

In some embodiments, the mobile workstation 100 is configured to allow the battery 134 to be replaced with a fully charged battery 134 without turning off the workstation, making the battery hot-swappable. This leads to increased efficiency because, when the battery 134 needs to be replaced, the electronic components powered by the battery 134 do not need to be turned off to replace the battery 134. Thus, the mobile workstation 100 may be configured for battery replacement without turning off the mobile workstation 100. In some embodiments, the base 102 is configured to hold two batteries 134.

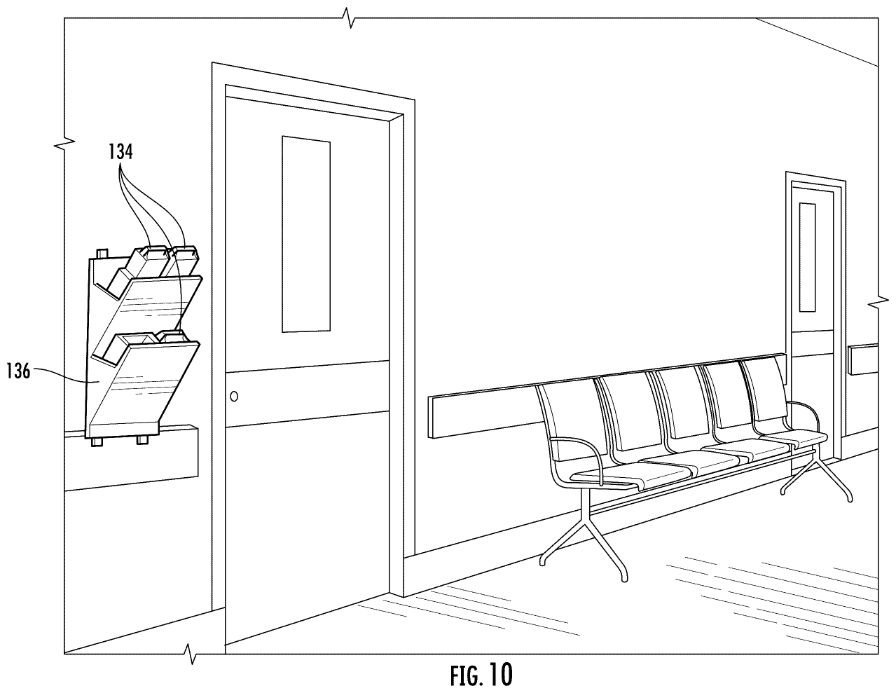

When implemented in a hospital setting or a similar setting, the mobile workstation 100 may be supported by charging stations 136 that are available to the user to charge the batteries 134. By providing a location close to the user, efficiency is further increased because the batteries 134 can be accessed easily and quickly as needed. Thus, as shown in FIG. 10, a charging station 136 may be located to be available every 10 rooms. Other numbers of rooms may also be implemented. Thus, the healthcare professional does not need to travel far to retrieve a fully charged battery 134 and place the depleted battery 134 on the charging station 136.

The mobile workstation 100 is intended for use by nursing staff to carry out various patient care tasks, but its applications are not limited to this. The unique design elements, including the extended stem 104 and the base 102, versatile tabletop 106, electronic attachments, and secure storage options make the mobile workstation 100 an efficient tool for users and healthcare providers, improving their ability to deliver high-quality patient care. The compact and lightweight design provides substantial space-saving opportunities and allows the workstation 100 to be easily maneuverable within healthcare facilities, enabling it to navigate tight spaces and be positioned close to patients.

In terms of hygiene, traditional WoWs often have numerous crevices that are difficult to clean, posing a risk for infection control. In some embodiments, the mobile workstation 100 is designed with minimal crevices, making it significantly easier to wipe down and disinfect, thus maintaining higher hygiene standards and reducing the risk of infection.

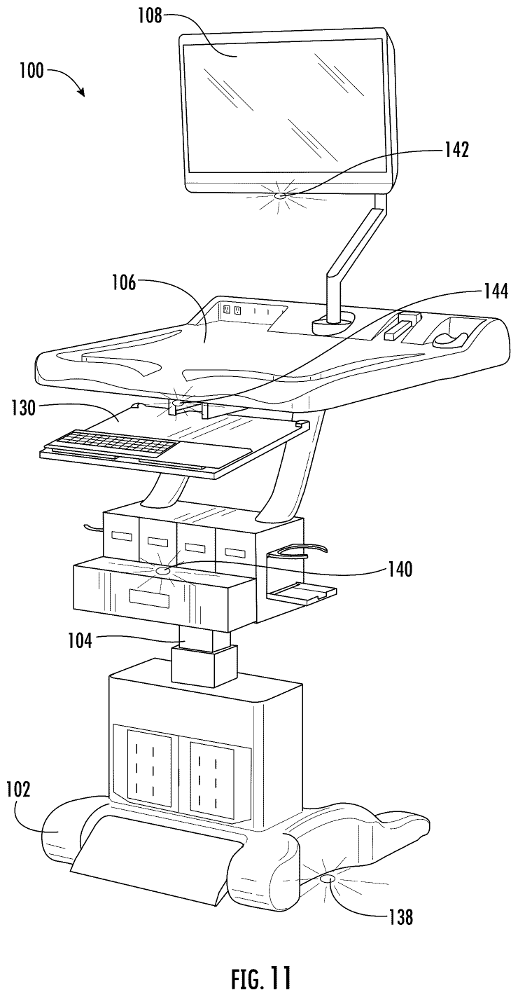

As shown in FIG. 11, the mobile workstation 100 may be equipped with features that facilitate use of the mobile workstation 100 in low-light situations, such as in a hospital room where a patient is sleeping. In some embodiments, the base 102 has a base light 138 is configured to illuminate the surroundings of the base 102. This helps the user to avoid obstacles when positioning the workstation 100. In some embodiments, the stem 104 has a storage light 140 configured to illuminate an interior of the plurality of compartments 118. This helps to illuminate the medical supplies stored inside. In some embodiments, the monitor has a table light 142 configured to illuminate the tabletop 106. This helps the user to be able to see papers, machines and tools, medications, or other items that are being used on the tabletop 106. In some embodiments, the mobile workstation 100 comprises a keyboard light 144 configured to illuminate the keyboard 132. The keyboard light 144 may be mounted to the tabletop 106, may be positioned on the keyboard 132 to backlight the keys of the keyboard 132, or may be positioned in another location where the keyboard light 144 can illuminate the keyboard 132. This allows the user to see the keyboard 132 clearly and facilitate typing on the keyboard 132. Any of the lights disclosed herein may be configured to turn on when the ambient light level surrounding the light lowers past a predetermined threshold. Similarly, any of the lights disclosed may be configured to automatically turn on and off in response to other changing conditions. For example, the table light 142 may automatically turn on when light levels are low. As another example, the storage light(s) 140 may turn on when one of the plurality of compartments 118 is opened.

The innovative design for the mobile workstation 100 has significant commercial potential because it addresses common challenges faced by healthcare professionals, leading to improved workflow efficiency and patient care outcomes. Its compact and ergonomic design is ideal for use in various medical and similar facilities, making it a valuable upgrade to existing counterparts within the healthcare industry. By providing a streamlined and operator-friendly solution, embodiments of the mobile workstation 100 have the potential to become an essential tool for healthcare providers.

The mobile workstation 100 has distinct advantages over WoWs that exist within the current healthcare market by addressing common WoW issues more effectively and greatly improving upon the functionality of such devices.

Embodiments of the mobile workstation 100 that have a stem 104 and a U-shaped, butterfly-style base 102 optimize space utilization and slide seamlessly beneath objects such as hospital beds. The upper portion 116 of the stem 104 supports a flowing tabletop 106, designed to facilitate various tasks and reduce the need for frequent movement. Additionally, the mobile workstation 100 includes versatile storage options with lockable and open compartments, providing secure and accessible storage for medical supplies and miscellaneous items. The mobile workstation 100 also lacks unnecessary crevices which makes it relatively easy to disinfect. Cordless electronic attachments supported by a long-lasting battery eliminate the need for frequent recharging, ensuring uninterrupted usage. The offset, highly maneuverable monitor 108 does not obstruct nurse-patient interaction, allowing nurses to connect better with their patients. Thus, this innovative design aims to enhance the workflow, physical comfort, and overall efficiency of healthcare professionals, supporting them in delivering optimal patient care and improving daily operations within healthcare environments.

The present disclosure addresses critical issues found in traditional WoWs and is a comprehensive upgrade over current technologies as a solution that significantly improves the efficiency, ergonomics, and overall functionality of mobile workstations in healthcare environments. However, the mobile workstation 100 is not limited to such settings. This innovative design not only improves the workflow of healthcare professionals but also leads to better patient care outcomes in ways that conventional WoWs do not.

Currently, the market for mobile workstations is populated by several established brands, such as Ergotron, Capsa Healthcare, and Rubbermaid Healthcare, which offer a variety of WoWs with different features and capabilities. However, these existing solutions fall short when trying to address the full spectrum of challenges faced by healthcare professionals, particularly in regard to space optimization, ease of cleaning, and facilitating nurse-patient interaction.

The present disclosure has substantial commercial potential in the healthcare industry, particularly in hospitals, clinics, and long-term care facilities where efficient and ergonomic workstations are critical for nursing staff, but is not limited to such areas. The design's unique features, such as the offset, highly-maneuverable monitor 108, U-shaped base 102, flowing tabletop 106, and minimal crevices, provide significant advantages over traditional mobile workstations. These improvements can lead to enhanced nurse-patient interactions, optimized space utilization, increased physical comfort, and improved hygiene standards, all of which are crucial in modern healthcare environments.

Embodiments of the present disclosure can be developed into a range of products catering to different healthcare settings and needs. This includes standard mobile workstations designed for general use in hospitals and clinics, specialized units that are tailored for specific departments such as emergency rooms, intensive care units, and surgical departments, compact versions for use in highly constrained spaces like small clinics or patient rooms, and customizable workstations that are configured to allow facilities to configure storage and electronic attachment options based on their specific requirements.

By addressing deficiencies found in traditional mobile workstations, embodiments of the present disclosure provide a superior alternative to current market offerings and set a new standard for mobile workstations in healthcare. The innovative design can significantly reduce nurse burnout and injuries, improve patient satisfaction, and maintain higher hygiene standards, making it an attractive option for healthcare facilities looking to upgrade their existing equipment. Given the industry's increasing focus on improving healthcare delivery, as well as the ongoing need for efficient, ergonomic tools in medical environments, the commercial potential for embodiments of the present disclosure is considerable.

Beyond healthcare, the mobile workstation 100 can be adapted for use in several other industries, offering similar benefits to mobility, space optimization, and ergonomic design. Potential applications include for managing registration desks, information booths, and mobile command centers at large events, in classrooms and laboratories for mobile teaching stations or student workstations, in hotels and restaurants for mobile concierge services, room service management, and check-in/check-out stations, for point-of-sale systems, inventory management, and customer service stations, and in research laboratories, pharmaceutical labs, and diagnostic labs for managing samples, documentation, and equipment.

The versatile and innovative design of the mobile workstation 100 makes it a suitable fit for a wide range of industries, boasting significant improvements to efficiency, ergonomics, and space utilization across various applications when compared to similar products. By addressing the challenges of mobility, space constraints, and ergonomic design that plague conventional mobile workstation solutions, embodiments of the present disclosure have the potential to revolutionize work environments far beyond healthcare.

It will be understood that implementations of the mobile workstation include but are not limited to the specific components disclosed herein, as virtually any components consistent with the intended operation of various mobile workstations may be utilized. Accordingly, for example, it should be understood that, while the drawings and accompanying text show and describe particular mobile workstation implementations, any such implementation may comprise any shape, size, style, type, model, version, class, grade, measurement, concentration, material, weight, quantity, and/or the like consistent with the intended operation of mobile workstation.

The concepts disclosed herein are not limited to the specific mobile workstations shown herein. For example, it is specifically contemplated that the components included in particular mobile workstations may be formed of any of many different types of materials or combinations that can readily be formed into shaped objects and that are consistent with the intended operation of the mobile workstation. For example, the components may be formed of: rubbers (synthetic and/or natural) and/or other like materials; glasses (such as fiberglass), carbon-fiber, aramid-fiber, any combination therefore, and/or other like materials; elastomers and/or other like materials; polymers such as thermoplastics (such as ABS, fluoropolymers, polyacetal, polyamide, polycarbonate, polyethylene, polysulfone, and/or the like, thermosets (such as epoxy, phenolic resin, polyimide, polyurethane, and/or the like), and/or other like materials; plastics and/or other like materials; composites and/or other like materials; metals, such as zinc, magnesium, titanium, copper, iron, steel, carbon steel, alloy steel, tool steel, stainless steel, spring steel, aluminum, and/or other like materials; and/or any combination of the foregoing.

Furthermore, mobile workstations may be manufactured separately and then assembled together, or any or all of the components may be manufactured simultaneously and integrally joined with one another. Manufacture of these components separately or simultaneously, as understood by those of ordinary skill in the art, may involve 3-D printing, extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled or removably coupled with one another in any manner, such as with adhesive, a weld, a fastener, any combination thereof, and/or the like for example, depending on, among other considerations, the particular material(s) forming the components.

In places where the description above refers to particular mobile workstation implementations, it should be readily apparent that a number of modifications may be made without departing from the spirit thereof and that these implementations may be applied to other implementations disclosed or undisclosed. The presently disclosed mobile workstations are, therefore, to be considered in all respects as illustrative and not restrictive.

We claim:

1. A mobile workstation, comprising:
a base configured to support and stabilize the mobile workstation in an upright position, wherein the base has a butterfly-style shape and comprises a base light configured to illuminate the surroundings of the base;
an extendable stem comprising:
a lower portion extending up from the base offset towards a front edge of the base;
a storage attachment mounted on the lower portion, the storage attachment having a plurality of compartments configured to store medical supplies and at least one storage light configured to illuminate an interior of the plurality of compartments; and
an upper portion extending up from the storage attachment angled away from the front edge of the base;
a tabletop positioned on the upper portion of the extendable stem;
a monitor extending up from the tabletop and having a table light configured to illuminate the tabletop;
a keyboard tray suspended below the tabletop configured to support a keyboard operatively coupled to the monitor; and
a rechargeable battery positioned in the base and configured to power the workstation, wherein the workstation is configured for battery replacement without turning off the workstation.

2. The mobile workstation of claim 1, wherein the workstation is designed with minimal crevices.

3. The mobile workstation of claim 1, wherein the workstation comprises a keyboard light configured to illuminate the keyboard.

4. The mobile workstation of claim 1, wherein the base is configured to slide at least partially beneath a hospital bed.

5. The mobile workstation of claim 4, wherein the upper portion of the extendable stem is angled such that when the base slides beneath the hospital bed, the tabletop is positioned above the hospital bed.

6. The mobile workstation of claim 1, wherein a position of the monitor with respect to the tabletop is adjustable.

7. A mobile workstation, comprising:
a base configured to support and stabilize the mobile workstation in an upright position, wherein the base has a butterfly-style shape;
an extendable stem comprising:
a lower portion extending up from the base offset towards a front edge of the base;
a storage attachment mounted on the lower portion, the storage attachment having a plurality of compartments configured to store medical supplies; and
an upper portion extending up from the storage attachment angled away from the front edge of the base;
a tabletop positioned on the upper portion of the extendable stem;
a monitor extending up from the tabletop; and
a rechargeable battery positioned in the base and configured to power the workstation, wherein the workstation is configured for battery replacement without turning off the workstation.

8. The mobile workstation of claim 7, wherein the base comprises a base light configured to illuminate the surroundings of the base.

9. The mobile workstation of claim 8, wherein the base light is configured to turn on when an ambient light level lowers past a predetermined threshold.

10. The mobile workstation of claim 7, further comprising a keyboard tray suspended below the tabletop configured to support a keyboard operatively coupled to the monitor.

11. The mobile workstation of claim 7, wherein the storage attachment comprises a glove box holder mounted to an outer surface of the storage attachment.

12. The mobile workstation of claim 7, wherein the base is configured to slide at least partially beneath a hospital bed.

13. The mobile workstation of claim 12, wherein the upper portion of the extendable stem is angled such that when the base slides beneath the hospital bed, the tabletop is positioned above the hospital bed.

14. A mobile workstation, comprising:
a base configured to support and stabilize the mobile workstation in an upright position;
a stem extending up from the base offset towards a front edge of the base;
a tabletop positioned on top of the stem;
a monitor extending up from the tabletop; and
a rechargeable battery positioned in the base and configured to power the workstation, wherein the workstation is configured for battery replacement without turning off the workstation.

15. The mobile workstation of claim 14, wherein the stem comprises:
a lower portion extending up from the base offset towards the front edge of the base;
a storage attachment mounted on the lower portion, the storage attachment having a plurality of compartments configured to store medical supplies; and
an upper portion extending up from the storage attachment angled away from the front edge of the base.

16. The mobile workstation of claim 14, wherein the base has a butterfly-style shape.

17. The mobile workstation of claim 14, wherein the base comprises a base light configured to illuminate the surroundings of the base.

18. The mobile workstation of claim 17, wherein the base light is configured to turn on when an ambient light level lowers past a predetermined threshold.

19. The mobile workstation of claim 14, wherein the base is configured to slide at least partially beneath a hospital bed.

20. The mobile workstation of claim 19, wherein the stem comprises an upper portion angled away from the front edge of the base such that when the base slides beneath the hospital bed, the tabletop is positioned above the hospital bed.
